(19) United States
(12) Patent Application Publication
(10) Pub. No.: US 2010/0036293 A1
(43) Pub. Date: Feb. 11, 2010

(54) HIFU treatment probe

(76) Inventors: Scott Isola, Deer Park, NY (US); Ronald R. Manna, Valley Stream, NY (US); Dan Voic, Cedar Grove, NJ (US)

Correspondence Address: R. Neil Sudol, 714 Colorado Avenue, Bridgeport, CT, 06605-1601, US

(21) Appl. No.: 12/462,401

(22) Filed: Aug. 3, 2009

Related U.S. Application Data
(60) Provisional application No. 61/137,962, filed on Aug. 5, 2008.
(60) Provisional application No. 61/188,734, filed on Aug. 12, 2008.

(51) Int. Cl. A61N 7/02

(52) U.S. Cl. 601/3

(57) ABSTRACT

A high-intensity focused ultrasound device with a translatory dive assembly and a rotary drive assembly mounted to a single-piece frame and disposed therewith inside a handle casing includes a focused ultrasound transducer driven by the drive assemblies via a pair of transducer shafts surrounded by an inner sleeve and outer sleeve that sandwich a bolus tube, the outer sleeve being slidably removable from atop the inner sleeve.

### CROSS-REFERENCE TO RELATED APPLICATION

This application claims the benefit under 35 U.S.C. §119(e) of U.S. Provisional Patent Application No. 61/137,962 filed 5 Aug. 2008 and U.S. Provisional Patent Application No. 61/188,734 filed 12 Aug. 2008.

### BACKGROUND OF INVENTION

This invention relates to high-intensity focused ultrasound (HIFU) for use in treating patients' internal tissue structures. More particularly the present invention relates to improvements in HIFU treatment probes.

High-intensity focused ultrasound (HIFU) devices are used in medicine to remove or neutralize malignant or diseased tissue. All high-intensity focused ultrasound (HIFU) devices currently on the market include a therapy transducer, a diagnostic transducer and a computer controlled electrical signal generator with integrated diagnostic systems. In practice, both the diagnostic and therapy transducers are allowed two degrees of freedom. One degree is longitudinal with respect to the axis of the device and the second degree of freedom is radial or in an arc with respect to the axis. This radial motion is also called sector motion.

Current HIFU treatment probes are difficult to sterilize particularly in the event that the bolus breaks while the distal end of the instrument is in contact with a patient's tissues. The bolus is an expandable chamber that contains the HIFU transducer. The bolus is expanded during a surgical procedure to enable the transmission of ultrasonic pressure waves into the patient from the transducer.

A prior art HIFU treatment probe 100 is depicted in FIGS. 1-4. The instrument includes a handle portion 102 and a shaft section 104. The handle portion 102 includes a handle casing or housing 106, a translatory drive assembly 108 mounted to a frame 110 inside the casing, and a rotary drive assembly 112 mounted to the frame and disposed inside the casing. Translatory drive assembly 108 includes a rotary motor 113 for linear motion generation, the rotary motor having an output shaft 114 connected to a spline shaft 116 via a flexible shaft coupler 118. Spline shaft 116 is journaled in a pair of rotary bearings 120 and 122 mounted to respective frame panels 124 and 126 in turn fixed to a plurality of longitudinally extending rails 128, 130 and 132 of multiple-piece frame 110. Spline shaft 116 carries a linear slide member 136 that is connected to a rear or proximal end of a hex drive shaft section 138 for longitudinally shifting that shaft section.

Rotary drive assembly 112 comprises a sector motor 140 and an encoder 142 with an encoder disk 144 for monitoring the angular excursion of a focused-ultrasound transducer 146 under the action of the rotary drive assembly. Motor 140 is mounted to frame 110 via a cylindrical frame extension 148. Translatory drive assembly 108 also includes an encoder (not shown) for monitoring the linear excursion of transducer 146 under the action of motor 113.

A transmission train 150 extends from translatory or linear drive assembly 108 and rotary drive assembly 112 to transducer 146. Transmission train includes hex drive shaft 138 and a forward or distal transducer drive shaft 152. A transducer shaft coupling 154 connects drive shaft sections 138 and 152 to one another. A shaft sleeve 155 mounted at a proximal end to the handle casing 106 surrounds distal transducer drive shaft 152 and is held in part by a pair of contiguous support cylinders 156 and 158 each provided at a proximal or rear end with a respective seal 160 and 162 (seal 162 is essentially impossible to clean). Three screws 164 fix cylinders 156 and 158 to one another. The heads of screws 164 (not separately designated) are disposed along a bolus chamber 166 that contains transducer 146. A rounded conical tip protector 168 is provided at the distal tip of sleeve 155.

### SUMMARY OF THE INVENTION

The present invention aims to provide an improved HIFU treatment probe of the above-described type. More particularly, the present invention contemplates a HIFU treatment probe that is readily sterilizable.

A high-intensity focused ultrasound device in accordance with the present invention comprises (i) a frame, (ii) a handle casing surrounding the frame, (iii) a translatory drive assembly mounted to the frame and disposed inside the casing, (iv) a rotary drive assembly mounted to the frame and disposed inside the casing, (v) a focused ultrasound transducer, (vi) a transmission train including at least one transducer shaft operatively connected at an upstream or input end to the translatory drive assembly and the rotary drive assembly and at a downstream end to the transducer, (vii) a shaft sleeve assembly mounted to the handle casing and surrounding the shaft, and (viii) a bolus tube attached to the shaft sleeve assembly and surrounding the transducer. The shaft sleeve assembly includes an inner sleeve and an outer sleeve disposed over the inner sleeve, the outer sleeve being slidably removable from atop the inner sleeve. A proximal or handle end portion of the bolus tube is sandwiched between the inner sleeve and the outer sleeve.

The shaft sleeve assembly or shaft housing of the present invention eliminates the need for shrink tubing. The outer sheath or sleeve may be made of stainless steel, which is impervious to conventional steam sterilization processes.

The shaft sleeve assembly or housing may include at least one support cylinder disposed inside the inner sleeve, the cylinder having a distal end face which bounds on a bolus chamber containing the transducer. The cylinder is formed at the end face with a seal about the transducer shaft. Preferably, the distal end face of the support cylinder is smooth and provided with a minimum of apertures consisting of only two openings for liquid flow into and out of the bolus chamber and an opening traversed by the transducer shaft. Thus, distal end face of the sleeve support cylinder is free of screws and screw heads. In addition, the bolus chamber is preferably free of temperature sensors.

This construction essentially eliminates obstructions in the bolus chamber and facilitates the cleaning of the device. The bolus chamber being essentially free of structures that would trap blood and organic contaminants from a patient promotes cleaning and sterilization.

The cylinder is preferably one of two support cylinders spaced longitudinally from one another along the transducer shaft. The other of the two support cylinders is likewise provided in a distal end surface with a seal about the transducer shaft. The sleeve or shaft housing construction of the present invention permits the removal of the outer and inner sleeves and enables access to the space between the two support cylinders for cleaning purposes.

Pursuant to another feature of the present invention, the translatory drive assembly includes a rotary output shaft assembly having a single bearing. The bearing is disposed on the frame at a forward or distal end of the rotary output shaft assembly, while the translatory drive assembly includes a motor mounted to a rear or proximal end of the frame. The provision of a single bearing (elimination of a rear bearing) facilitates assembly of the device by accommodating misalignment.

Another feature of a HIFU probe in accordance with the present invention that facilitates assembly is the use of a single piece frame in the handle. The frame supports the translatory drive assembly. A single piece reduces the necessity for fine tolerance manufacture of multiple frame pieces.

A prior art HIFU device incorporates a liquid circulation system including an inlet coupling and an outlet coupling on the casing and tubing extending between the inlet coupling and a bolus chamber and between the bolus chamber and the outlet coupling, where the transducer is disposed in the bolus chamber. Pursuant to the present invention, a thermocouple is disposed in the handle casing in line between the bolus chamber and the outlet coupling. Thus, the temperature of the liquid in the bolus may be adequately monitored without having a temperature sensor in the bolus chamber.

### BRIEF DESCRIPTION OF THE DRAWINGS

FIG. 1 is a longitudinal cross-sectional view of a prior art HIFU treatment probe.

FIG. 2 is an isometric view of a multi-piece handle frame including a linear or translatory drive assembly, in the prior art HIFU probe of FIG. 1.

FIG. 3 is an exploded view of a shaft section of the probe of FIG. 1.

FIG. 4 is a perspective view of the portion of the shaft of FIG. 3, in an assembled configuration.

FIG. 5 is a longitudinal cross-sectional view of a HIFU treatment probe in accordance with the present invention.

FIG. 6 is an enlarged detail, in cross-section, taken from area VI in FIG. 5.

FIG. 7 is a front side perspective view of internal components of the HIFU treatment probe of FIG. 5.

FIG. 8 is a partially exploded front perspective view, on a slightly larger scale, of the internal components of FIG. 7.

FIG. 9 is a rear side perspective view of a rear or proximal portion of the internal components of FIGS. 7 and 8.

FIG. 10 is a rear perspective view, on a substantially larger scale, of a sleeve or sheath support assembly shown in FIGS. 5, 7, and 8.

FIG. 11 is a front perspective view, on a substantially larger scale, of the sleeve or sheath support assembly shown in FIGS. 5, 7, 8, and 10.

FIG. 12 is a perspective view, on a reduced scale, of the HIFU treatment probe of FIG. 5, showing an outer sleeve or sheath removed.

FIG. 13 is a perspective view, similar to FIG. 12, of the HIFU treatment probe of FIGS. 5 and 10, showing a sleeve or sheath assembly in a disassembled or exploded configuration.

FIG. 14 is an enlarged detail, in perspective, taken from area XIV in FIG. 13.

FIG. 15 is an enlarged detail, in perspective, taken from area XV in FIG. 12.

FIG. 16 is an enlarged detail, in perspective, taken from area XVI in FIG. 12.

FIG. 17 is a perspective view of a frame and drive assembly shown in FIGS. 5 and 8.

FIG. 18 is an exploded perspective view of a portion of the frame and drive assembly of FIGS. 5, 8, and 17.

FIG. 19 is an exploded perspective view of another portion of the frame and drive assembly of FIGS. 5, 8, and 17.

### DETAILED DESCRIPTION

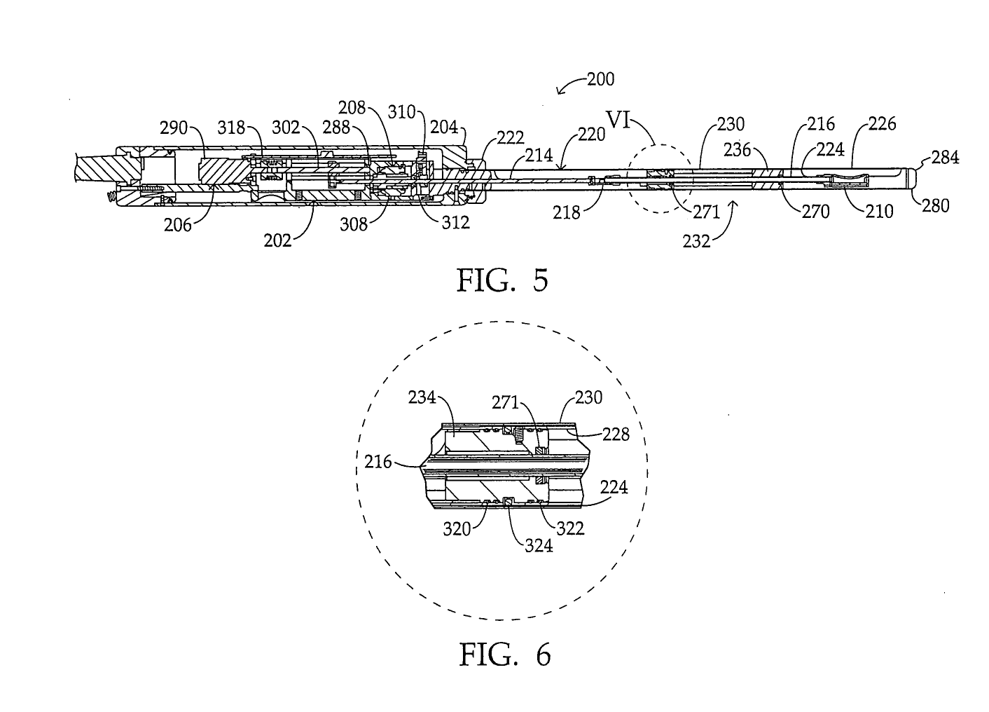

As depicted in FIG. 5, a high-intensity focused ultrasound device 200 comprises a frame 202, a handle casing or housing 204 surrounding the frame, a translatory drive assembly 206 mounted to the frame and disposed inside the casing, a rotary drive assembly 208 mounted to the frame and disposed inside the casing, and a focused ultrasound transducer 210. Translatory drive assembly 206 and rotary drive assembly 208 are operatively connected to transducer 210 via a mechanical transmission train 212 including an upstream or proximal transducer shaft section 214 and a downstream of distal drive shaft section 216. The transducer drive sections 214 and 216 are linked to one another via a transducer shaft coupling 218. Upstream or proximal transducer drive shaft section 214 is operatively connected at an upstream or input end to translatory drive assembly 206 and rotary drive assembly 208, while downstream or distal transducer shaft section 216 is connected at a forward or distal end to transducer 210.

A shaft sleeve assembly 220 is mounted to handle casing 204 via an outer-sleeve attachment nut 222 and surrounds transducer shaft sections 214 and 216. A bolus tube 224 (FIG. 13) is attached to shaft sleeve assembly 220 and contains transducer 210 in a bolus chamber 226. Shaft sleeve assembly 220 includes an inner sleeve 228 and an outer sleeve 230 slidably disposed over the inner sleeve. A proximal or handle end portion (not separately designated) of bolus tube 224 is sandwiched between inner sleeve 228 and outer sleeve 230 as best illustrated in FIG. 6.

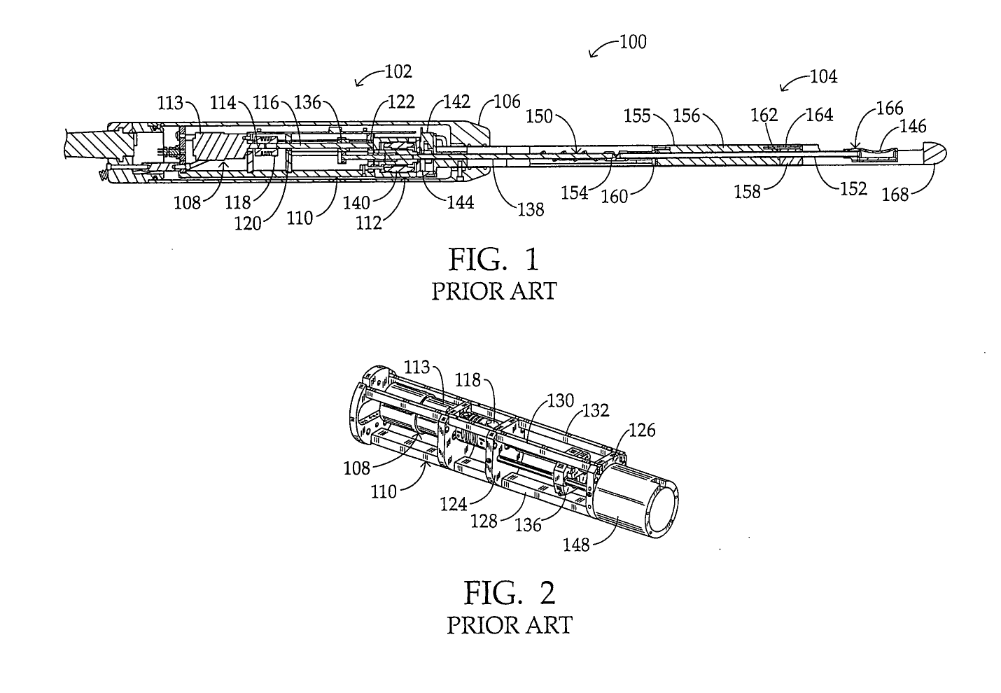

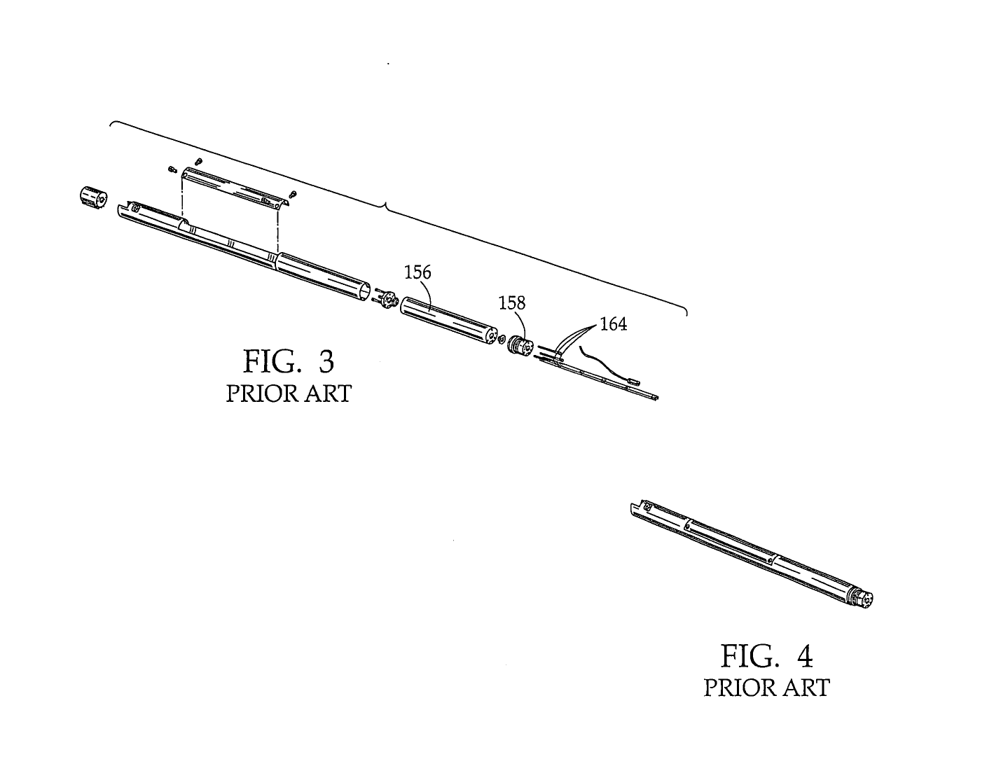

Shaft sleeve assembly or shaft housing 220 eliminates the need for shrink tubing that exists in the prior art HIFU treatment probe 100 depicted in FIGS. 1-4. Outer sheath or sleeve 230 may be made of stainless steel.

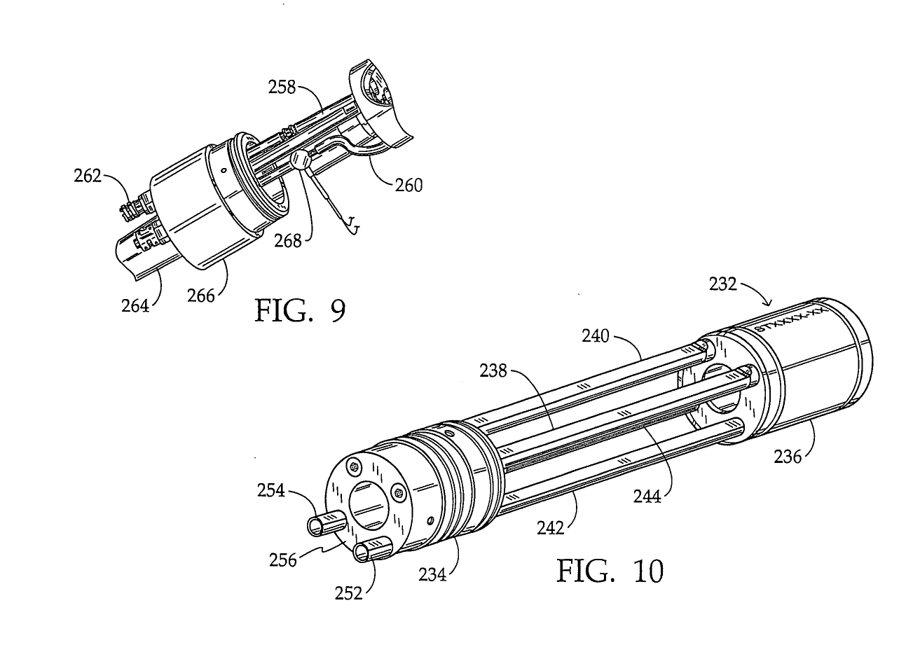

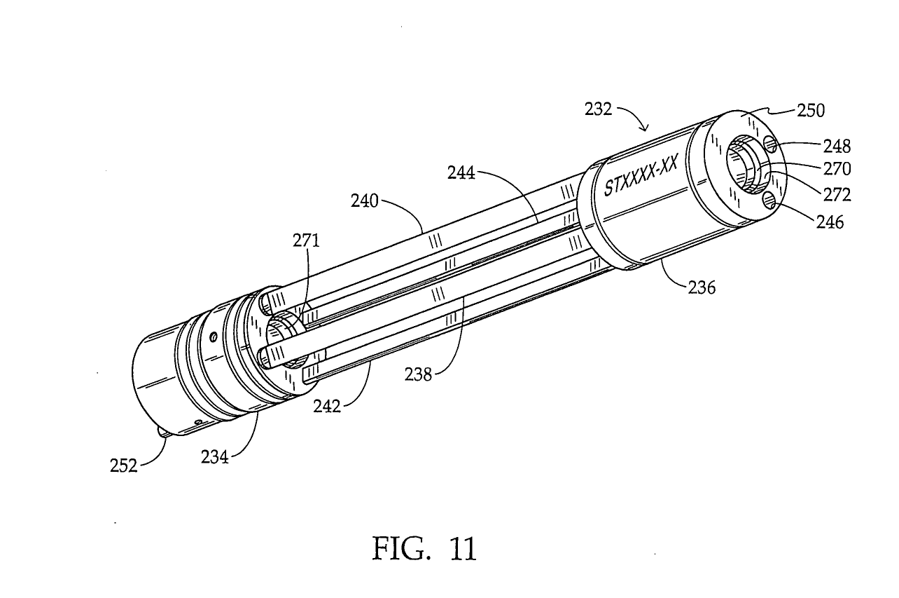

Shaft sleeve assembly or housing 220 includes a sleeve or sheath support assembly 232, best depicted in FIGS. 10 and 11. Sleeve or sheath support assembly 232 includes a proximal support cylinder 234 and a distal support cylinder 236 that are spaced from one another and rigidly interconnected by a pair of rods 238 and 240 and a pair of tubes 242 and 244. Tubes 242 and 244 communicate on a distal side with bolus chamber 226 via respective end openings 246 and 248 in a distal end face 250 of distal support cylinder 236, distal end face forming a proximal-side boundary of the bolus chamber. Tubes 242 and 244 communicate on a proximal side with respective nipples 252 and 254 that project from a proximal end face 256 of proximal support cylinder 234. Nipples 252 and 254 are connected to respective hoses or tubing segments 258 and 260 (FIG. 9) that extend through handle casing 204 and communicate with respective coupling ports 262 and 264 on a rear end cap 266 of the handle casing. These various components define a fluid flow path that extends in a distal direction from inlet coupling port 262 and through hose or tubing segment 258, nipple 252, tube 242, and opening 246 to bolus chamber 226 and back in a proximal direction from the bolus chamber through opening 248, tube 244, nipple 254 and hose or tubing segment 260 to outlet port 264. A thermocouple 268 is disposed in handle casing 208 in line with hose or tubing segment 260 and outlet port 264, for monitoring the temperature of the liquid flowing from bolus chamber 226. In contrast with the prior art model (FIGS. 1-4), there is no temperature sensor in bolus chamber 226. A liquid such as sterile water is circulated along the flow path through bolus chamber 226 for purposes of enabling bolus distension, for effectuating ultrasonic wave transmission into organic tissue, and for cooling transducer 210.

Distal cylinder 236 is formed at end face 250 with a seal 270 (FIG. 11) about transducer shaft section 216. Distal end face 250 is smooth and provided with a minimum of apertures, namely, openings 246 and 248 for liquid flow into and out of bolus chamber 226 and an opening 272 traversed by transducer shaft section 216. Thus, distal end face of sleeve support cylinder 236 is free of screws and screw heads, in contrast to the prior art treatment probe of FIGS. 1-4. As described above, bolus chamber 226 is free of temperature sensors, thermocouple 268 being disposed inside handle casing 204. Bolus chamber 226 is therefore essentially empty of obstructions that could trap blood and organic contaminants (in the event of a bolus tube rupture during an ultrasonic ablation procedure. In addition, seal 270 (FIG. 11) is at the front of sleeve support cylinder 236, which substantially facilitates cleaning of the seal.

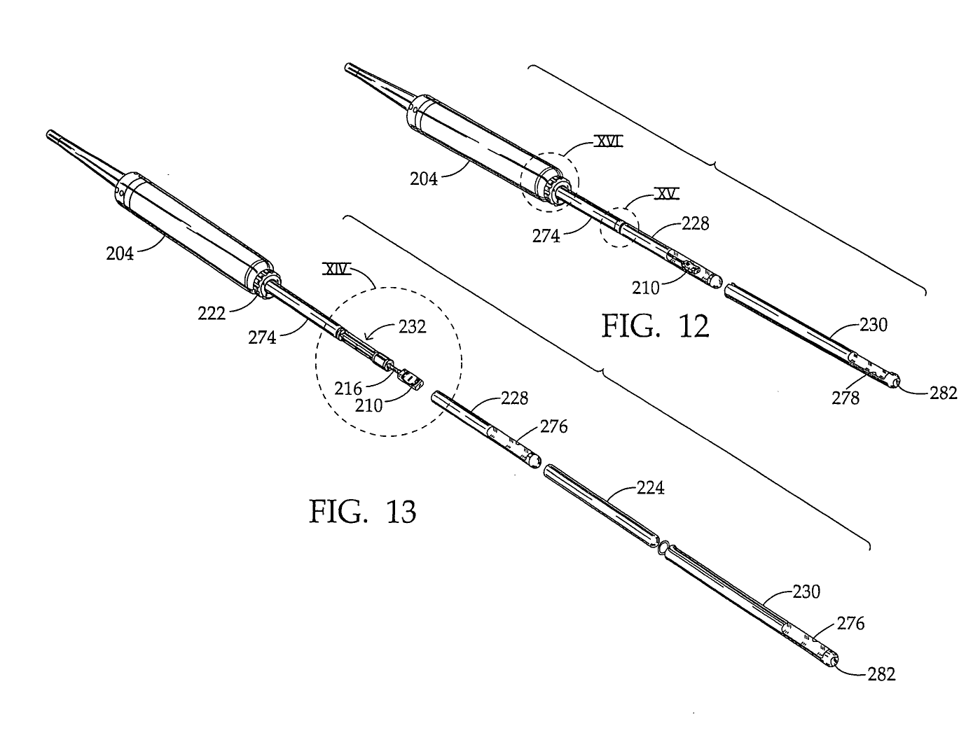

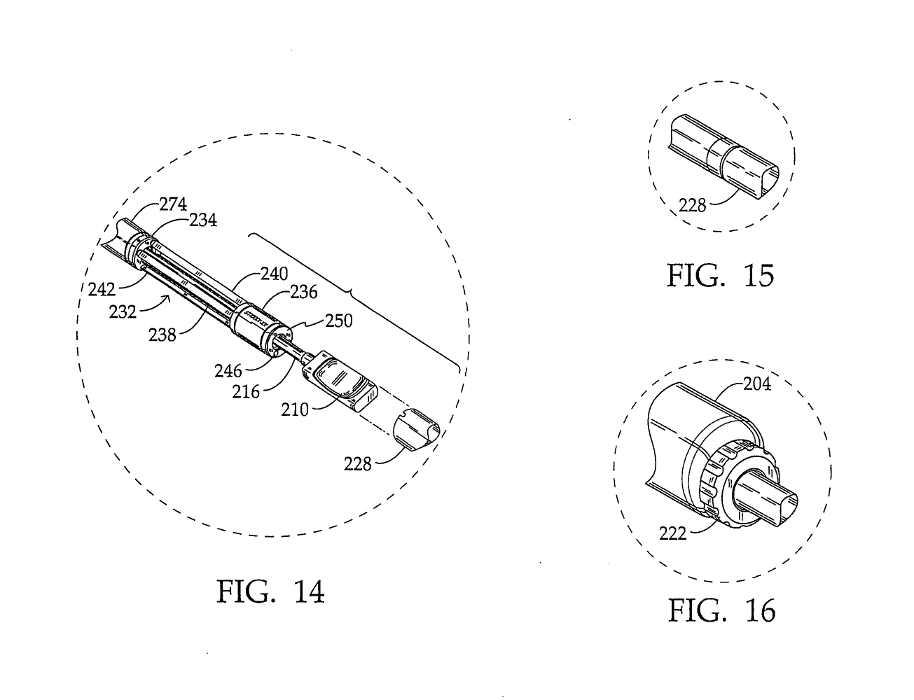

Inner sleeve 228 is a most distal of two inner sleeve sections 228 and 274, where the proximal sleeve 274 is attached to handle casing 204. As depicted in FIGS. 12-15, proximal sleeve support cylinder 234 is disposed inside a distal end of proximal inner sleeve 274 and inside a proximal end section of distal inner sleeve section 228. Distal inner sleeve section 228 may optionally slide over a distal end of proximal inner sleeve section 274. As depicted in FIG. 13, bolus tube 224 is slid over distal inner sleeve section 228 after that sleeve section has been secured to proximal inner sleeve section 274 at proximal support cylinder 234. Then outer sleeve 230 is slidably and removably inserted over bolus tube 224 and distal inner sleeve section 228 and coupled to handle casing 204 by means of attachment nut 222. Inner sleeve section 228 and outer sleeve 230 are provided at distal ends with elongate lateral windows 276 and 278 (FIG. 13) that are alignable with one another and with transducer 210. Bolus tube 224 is expandable out through the aligned windows 276 and 278 to form an effective pressure-wave-transmitting contact with target organic tissues of a patient. The bolus rolls over outer sleeve 230 without the need for shrink tubing.

The sleeve construction of FIG. 13, wherein stainless outer sleeve 230 is easily and quickly removably from inner sleeve section 228, facilitates cleaning and bolus tube replacement. Access is thus provided to the space between support cylinders 234 and 236.

At its distal tip ultrasound probe 200 is provided with a tip protector 280 (FIG. 5) that is partially inserted into an aperture 282 at the distal end of outer sleeve 230 (FIGS. 12 and 13). Tip protector 280 has a flat end face 284 that occupies reduced space relative to a rounded conical tip protector of the prior art (see FIGS. 104).

As depicted in FIG. 5, translatory drive assembly 206 includes a rotary output shaft assembly 286 having a single bearing 288. Bearing 288 is disposed on frame 202 at a forward or distal end of rotary output shaft assembly 286. Translatory drive assembly 206 includes a motor 290 mounted to a rear or proximal end of frame 202. The provision of a single bearing 288 (elimination of a rear bearing) facilitates assembly of the device by accommodating misalignment.

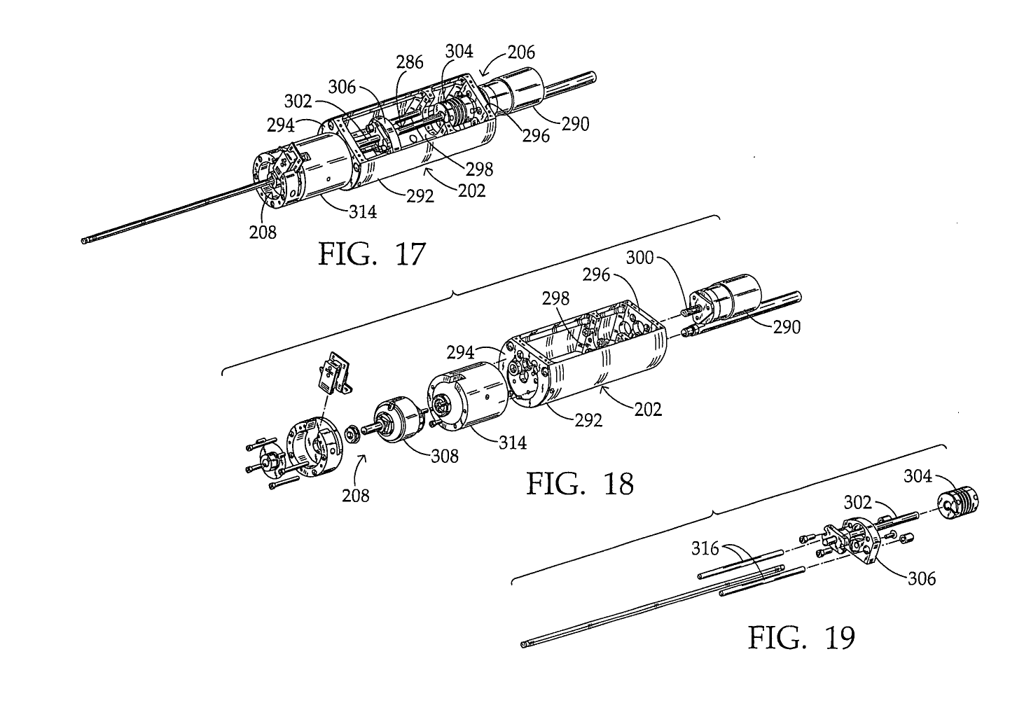

As shown in FIGS. 17 and 18, frame 202 is a single molded or machined piece comprising a sectioned cylindrical wall 292, a pair of sectioned or truncated circular end panels 294 and 296 and a middle panel or brace 298 all integral with cylindrical wall 292. Motor 290 has an output shaft 300 connected to a spline shaft 302 via a flexible shaft coupler 304. Spline shaft 304 is journaled at a forward or distal end in bearing 288, which is disposed in end panel 294. Spline shaft 304 carries a linear slide member 306 that is connected to a rear or proximal end of hex transducer drive shaft section 214 for longitudinally shifting that shaft section and consequently shaft section 216 and transducer 210. Linear slide member 306 moves along a pair of longitudinal guide rods 316 that are fixed to frame panels 294 and 298.

Rotary drive assembly 208 comprises a sector motor 308 and an encoder 310 with an encoder disk 312 (FIG. 5) for monitoring the angular excursion of focused-ultrasound transducer 210 under the action of the rotary drive assembly. Motor 308 is mounted to frame 202 via a cylindrical frame extension 314. Translatory drive assembly 206 also includes an encoder (not shown) for monitoring the linear excursion of transducer 210 under the action of motor 290.

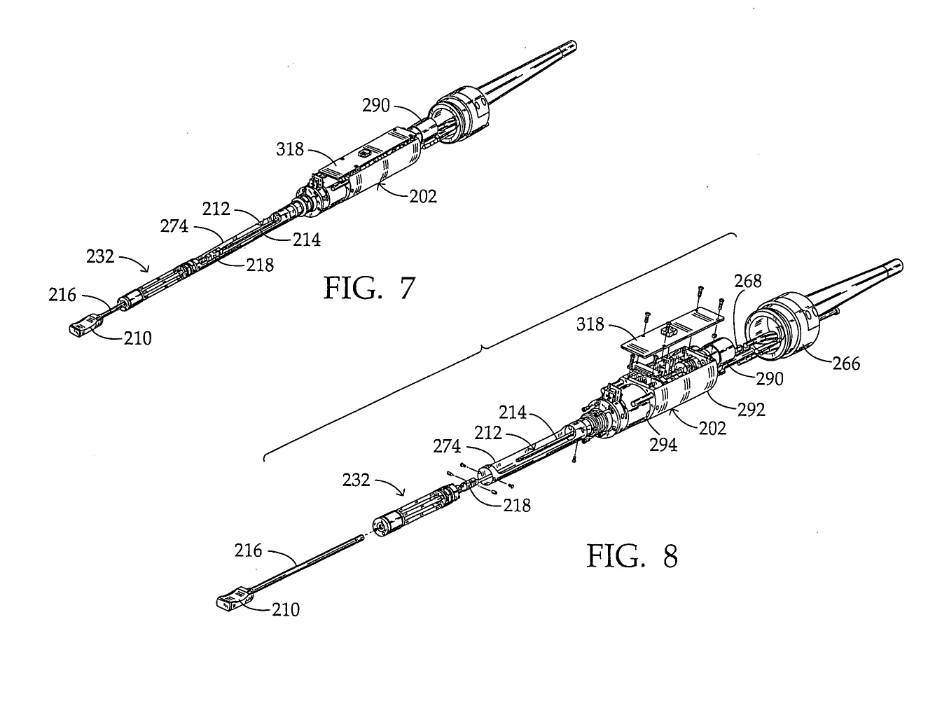

A printed circuit board 318 is fastened to frame 202 (FIGS. 5, 7 and 8) for controlling translatory drive assembly 206 and rotary drive assembly 208 pursuant to programmed instructions from an operator.

As depicted in FIGS. 6 and 11, proximal cylinder 234 is provided at a distal side with a shaft seal 271. As shown in FIG. 6, a first pair of O-ring seals 320 is provided at a rear end of proximal cylinder 234 for sealingly engaging proximal inner sleeve section 274, while a second pair of O-ring seals 322 is provided at a forward end of proximal cylinder 234 for sealingly engaging distal inner sleeve section 228. Another O-ring seal 324 engages cylinder 234 and bolas tube 224.

Although the invention has been described in terms of particular embodiments and applications, one of ordinary skill in the art, in light of this teaching, can generate additional embodiments and modifications without departing from the spirit of or exceeding the scope of the claimed invention. Accordingly, it is to be understood that the drawings and descriptions herein are proffered by way of example to facilitate comprehension of the invention and should not be construed to limit the scope thereof.

What is claimed is:

1. A high-intensity focused ultrasound device comprising: a frame; a handle casing surrounding said frame; a translatory drive assembly mounted to said frame and disposed inside said casing; a rotary drive assembly mounted to said frame and disposed inside said casing; a focused ultrasound transducer; a transmission train including at least one transducer shaft operatively connected at an upstream or input end to said translatory drive assembly and said rotary drive assembly and at a downstream end to said transducer; a shaft sleeve assembly mounted to said handle casing and surrounding said shaft; and a bolus tube attached to said shaft sleeve assembly and surrounding said transducer, said shaft sleeve assembly including an inner sleeve and an outer sleeve disposed over said inner sleeve, said outer sleeve being slidably removable from atop said inner sleeve, a proximal or handle end portion of said bolus tube being sandwiched between said inner sleeve and said outer sleeve.

2. The device defined in claim 1 wherein said sleeve assembly includes at least one support cylinder disposed inside said inner sleeve, said cylinder having a distal end face facing a bolus chamber containing said transducer, said cylinder being formed at said end face with a seal about said transducer shaft.

3. The device defined in claim 2 wherein said distal end face is smooth and provided with apertures consisting of only two openings for liquid flow into and out of said bolus chamber and an opening traversed by said transducer shaft.

4. The device defined in claim 3 wherein said distal end face is free of screws.

5. The device defined in claim 2 wherein said cylinder is one of two support cylinders spaced longitudinally from one another along said transducer shaft, another of said two support cylinders being provided in a distal end surface with a seal about said transducer shaft.

6. The device defined in claim 2 wherein said bolus chamber is free of temperature sensors.

7. The device defined in claim 1 wherein said translatory drive assembly includes a rotary output shaft assembly having a single bearing.

8. The device defined in claim 7 wherein said bearing is disposed on said frame at a forward or distal end of said rotary output shaft assembly, said translatory drive assembly including a motor mounted to a rear or proximal end of said frame.

9. The device defined in claim 1, further comprising a liquid circulation system including an inlet coupling and an outlet coupling on said casing and tubing extending between said inlet coupling and a bolus chamber and between said bolus chamber and said outlet coupling, said transducer being disposed in said bolus chamber, also comprising a thermocouple disposed in said handle casing in line between said bolus chamber and said outlet coupling.

10. The device defined in claim 1 wherein said frame is a single-piece frame.

11. The device defined in claim 1 wherein said inner sleeve and said outer sleeve are provided with windows aligned with said transducer.

12. The device defined in claim 1 wherein said transducer shaft includes at least two shaft segments connected to one another by a transducer shaft coupling.

13. A high-intensity focused ultrasound device comprising: a frame; a handle casing surrounding said frame; a translatory drive assembly mounted to said frame and disposed inside said casing; a rotary drive assembly mounted to said frame and disposed inside said casing; a focused ultrasound transducer; a transmission train including at least one transducer shaft operatively connected at an upstream or input end to said translatory drive assembly and said rotary drive assembly and at a downstream end to said transducer; a shaft sleeve assembly mounted to said handle casing and surrounding said shaft; and a bolus tube attached to said shaft sleeve assembly and surrounding said transducer, said shaft sleeve assembly including at least one support cylinder, said cylinder having a distal end face facing a bolus chamber containing said transducer, said cylinder being formed at said end face with a seal about said transducer shaft, said distal end face being smooth and provided with apertures including only two openings for liquid flow into and out of said bolus chamber and an opening traversed by said transducer shaft, said distal end face being free of screws.

14. The device defined in claim 13 wherein said bolus chamber is free of temperature sensors.

15. The device defined in claim 14, further comprising a liquid circulation system including an inlet coupling and an outlet coupling on said casing and tubing extending between said inlet coupling and a bolus chamber and between said bolus chamber and said outlet coupling, said transducer being disposed in said bolus chamber, also comprising a thermocouple disposed in said handle casing in line between said bolus chamber and said outlet coupling.

16. The device defined in claim 13 wherein said cylinder is one of two support cylinders spaced longitudinally from one another along said transducer shaft, another of said two support cylinders being provided in a distal end surface with a seal about said transducer shaft.

17. The device defined in claim 13 wherein said frame is a single-piece frame.

18. A high-intensity focused ultrasound device comprising: a single-piece frame; a handle casing surrounding said frame; a translatory drive assembly mounted to said frame and disposed inside said casing; a rotary drive assembly mounted to said frame and disposed inside said casing; a focused ultrasound transducer; a transmission train including at least one transducer shaft operatively connected at an upstream or input end to said translatory drive assembly and said rotary drive assembly and at a downstream end to said transducer; a shaft sleeve assembly mounted to said handle casing and surrounding said shaft; and a bolus tube attached to said shaft sleeve assembly and surrounding said transducer.
